(19) United States
(12) Patent Application Publication
(10) Pub. No.: US 2005/0250853 A1
(43) Pub. Date: Nov. 10, 2005

(54) Beta-tubulin inhibitors

(76) Inventors: Rene C. Gaudreault, Bernieres (CA); Jean Legault, Clermont-Ferrand (FR)

Correspondence Address: MEREK, BLACKMON & VOORHEES, LLC, 673 S. WASHINGTON ST., ALEXANDRIA, WV, 22314, US

(21) Appl. No.: 11/072,334

(22) Filed: Mar. 7, 2005

Related U.S. Application Data
(62) Division of application No. 09/741,814, filed on Dec. 22, 2000, now abandoned.
(60) Provisional application No. 60/171,615, filed on Dec. 23, 1999.

(51) Int. Cl. A61K 31/17

(52) U.S. Cl. 514/596

(57) ABSTRACT

Disclosed herein are β-tubulin inhibitors of formula I, prodrugs thereof and therapeutically acceptable salts thereof, 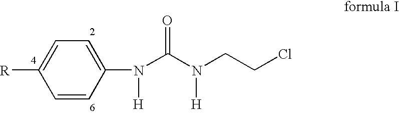 wherein R is selected from the group consisting of: t-butyl, i-propyl and sec-butyl and their use as anti-cancer cell proliferation agents.

### FIELD OF THE INVENTION

The present invention relates to anticancer molecules that inhibit β-tubulin and that illustrate lower systemic toxicity, higher therapeutic index and a lower capacity to induce resistant phenotypes, which would greatly improve chemotherapy. More specifically, the present invention is directed to the use of various derivatives of 1-aryl-3-(2-chloroethyl)ureas as β-tubulin inhibitors. The present invention is also concerned with a method of selectively attacking cancer cell key proteins by providing derivatives of 1-aryl-3-(2-chloroethyl)ureas having specific spatial configurations allowing these derivatives to dock inside cells at pre-selected sites.

### THE PRIOR ART

Cancer is a disease state characterized by the uncontrolled proliferation of genetically altered tissue cells. The deleterious effects of most anticancer agents, in combination with the occurrence tumor drug resistance, contribute to failure and relapse of the disease following initial responses to chemotherapy. There have been several chemotherapeutic approaches developed to target cancer including alkylating and anti-mitotic agents, anti-metabolites and anti-tumor antibiotics. Such therapeutic agents act preferentially on rapidly proliferating cells such as cancer cells.

Hormonal therapy using anti-estrogens or anti-androgens constitutes another method of attacking cancer cells.

Some 1-aryl3-(2-chloroethyl)urea derivatives (herein after referred to as “CEUs”) are known from U.S. Pat. Nos. 5,530,026 and 5,750,547 to the same assignee as the present application. More specifically 4-tBCEU, 4-iPCEU and 4-sBCEU are known, whose structures are as illustrated below:

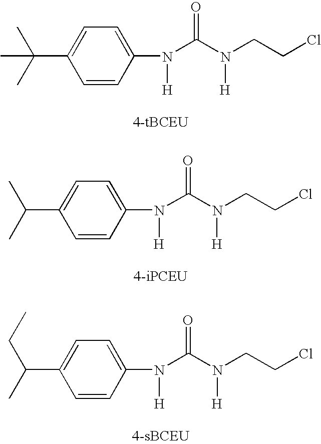

CEUs are also known from PCT application WO 0061546, also to the same assignee as the present application.

It is known that CEUs display affinity towards cancer cells, permeate the cell wall and provide a mild alkylating effect on cell components thereby killing the offending cell.

An object of the present invention is to provide CEU derivatives capable of inhibiting β-tubulin.

A further object of the present invention is to provide weak monoalkylating agents that are unreactive towards most cellular nucleophiles such as DNA, glutathion and glutathion reductase but capable of alkylating specific proteins bearing strong nucleophilic centers.

Yet another object of the present invention is to provide prodrugs of the β-tubulin inhibitors as well as inorganic salts thereof. As an example of prodrugs of the compounds of the present invention, the sulfone and sulfoxide derivatives are immediately contemplated by a skilled worker in the art. The sulfone and sulfoxide derivatives while not generally active will be activated once administered to the patient. The activation will occur when the prodrug is reduced to yield the corresponding alkylthio derivative, an active compound.

### SUMMARY OF THE INVENTION

The present invention provides for β-tubulin inhibitors of formula 1, prodrugs thereof and therapeutically acceptable salts thereof, 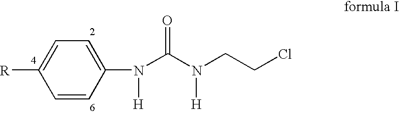
wherein R is selected from the group consisting of: t-butyl, i-propyl and s-butyl. The present invention also provides method of curtailing cancer cell proliferation using a medicament comprising a therapeutically effective amount of the compound of formula 1, prodrugs thereof or therapeutically acceptable salts thereof.

Further scope and applicability of the present invention will become apparent from the detailed description given hereinafter. It should be understood, however, that this detailed description is given by way of illustration only, since various changes and modifications within the spirit and scope of the invention will become apparent to those skilled in the art.

### BRIEF DESCRIPTION OF THE FIGURES

FIG. 1. Effect of CEU derivatives on cell cycle. Exponentially growing MDA-MB-231 cells were incubated in the absence or presence of 30 μM of either 4-tBCEU, 4-iPCEU or 4-sBCEU for 24 and 48 h, respectively, at 37° C. The cell cycle was evaluated using propidium iodide staining and flow cytometric analysis. Data are representative of three independent experiments.

FIG. 2. Effects of CEU derivatives on microtubule depolymerization. Exponentially growing MDA-MB-231 cells were incubated in the absence or the presence of 30 μM of CEU derivatives including (A) 4-tBCEU, 4-iPCEU or 4-sBCEU for 24 and 48 h respectively; (B) 2-ECEU, 3-ECEU or 4-ECEU for 24 and 48 h; and (C) 4-tBEU, 4-iPEU, 4-sBEU, 4-tBCPU, CEU or 4-methoxyCEU for 48 h. Free tubulin and microtubule fractions were isolated and analyzed by Western blot. Data are representative of three different experiments.

FIG. 3. Protein alkylation induced by treatment with [urea-¹⁴C]-4-tBCEU. (A) Post-nuclei protein extracts from MDA-MB-231 cells incubated with 30 or 100 μM of [urea-¹⁴C]-4-tBCEU for 12 and 24 h. Panel (B) shows electrophoretic separation of total (T), cytosolic (C) and insoluble (M) proteins, extracted from MDA-MB-231 cells treated with 30 μM [urea-¹⁴C]-4-tBCEU for 24 h. The protein extracts were analyzed by SDS-PAGE. The gels were transferred onto nitrocellulose membranes and [urea-¹⁴C]-4-tBCEU-labeled proteins were revealed by autoradiography. Data are representative of four different experiments. The arrow points to β-tubulin.

FIG. 4. Characterization of the [urea-¹⁴C]-4-tBCEU-labeled 50-kDa protein by 2-D gel electrophoresis. MDA-MB-231 cells were treated with 30 βM [urea-¹⁴C]-4-tBCEU for 48 h at 37° C. Total protein extracts were separated by 2-D electrophoresis. The gels were then transferred onto nitrocellulose membranes. (A) The presence of β-tubulin was revealed by immunoblotting; (B) [urea-¹⁴C]-4-tBCEU-labeled proteins were visualized by autoradiography. Data are representative of three different experiments.

FIG. 5. Kinetics of β-tubulin alkylation and microtubule disruption induced by [urea-¹⁴C]-4-tBCEU. Exponentially growing MDA-MB231 cells were incubated with 30 μM [urea-¹⁴C]-4-tBCEU for 0, 1, 4, 8, 12, 18, 24 and 48 h. (A) Free tubulin and microtubule fractions were isolated and analyzed by Western blotting; (B) [urea-¹⁴C]-4-tBCEU-labeled proteins were visualized by autoradiography. Data are representative of three different experiments.

FIG. 6. Identification of the site of alkylation of β-tubulin by CEU: competition with antimicrotubule agents. MDA-MB231 cells were incubated (A) for 48 h with 30 μM 4-tBCEU, 4-iPCEU or 4-sBCEU in the absence or presence of 5 μM taxol; (B) for 48 h with 30 μM 4-tBCEU or 4-sBCEU in the absence or presence of 5 μM colchicine (Col), 5 μM vinblastine (Vbl) or 100 μM iodoacetamide (lodo); (C) for 24 h with 5 μM colchicine, 5 μM vinblastine or 100 μM iodoacetamide, respectively, and followed by treatment with 100 μM EBI for 2 h. Total protein extracts were analyzed by SDS-PAGE and β-tubulin was revealed by immunoblotling. Data are representative of three different experiments.

FIG. 7. Localization of the site of alkylation by CEU on β-tubulin. Exponentially growing SK—N—SH cells were incubated in the absence or the presence of 30 μM and 100 μM [urea-¹⁴C]-4-tBCEU for 24 h and 48 h, respectively. Total protein extracts were separated electrophoretically on 10% polyacrylamide gels. (A) Western blots were performed and βIII-tubulin was revealed with a monoclonal antibody; (B) [urea-¹⁴C]-4-tBCEU-labeled proteins were visualized by autoradiography. Results are representative of two independent experiments.

FIG. 8. Hypothetical alkylation of β-tubulin by CEU.

### DETAILED DESCRIPTION OF PREFERRED EMBODIMENTS

Before describing the present invention in detail, it is to be understood that the invention is not limited in its application to the details of the examples described herein. The invention is capable of other embodiments and of being practiced in various ways. It is also to be understood that the phraseology or terminology used herein is for the purposes of description and not limitation.

Non standard abbreviations used in the paper are: CEU, 1-aryl-3-(2-chloroethyl)urea; 4-tBCEU, 4-tert-butyl[3-(2-chloroethyl)ureido]benzene; 4-iPCEU, 4-iso-propyl[3-(2-chloroethyl)ureido]benzene; 4-sBCEU, 4-sec-butyl[3-(2-chloroethyl)ureido]benzene; 2-ECEU, 2-ethyl[3-(2-chloroethyl)ureido]benzene; 3-ECEU, 3ethyl[3-(2-chloroethyl)ureido]benzene; 4-ECEU, 4-ethyl(3-(2-chloroethyl)ureido]benzene; 4-tBEU, 4-tert-butyl[3-(ethyl)ureido]benzene; 4-iPEU, 4-iso-propyl[3-(ethyl)ureido]benzene; 4-sBEU, 4-sec-butyl[3-(ethyl)ureido]benzene; 4-tBCPU, 4-tert-butyl[3-(2-chloropropyl)ureido]benzene; 4-methoxyCEU, 4-methoxy[3-(2-chloroethyl)ureido]benzene; EBI, N,N′-ethylenebis(iodoacetamide); PBS, phosphate-buffered saline.

Preparation of CEU Derivatives

The β-tubulin inhibitors of the present invention are easily prepared in generally good yields. The compounds are also easily purified by usual techniques such as chrystallization or liquid chromatography.

The following reaction sequence illustrates one general scheme of preparation of CEU derivatives of the present invention.

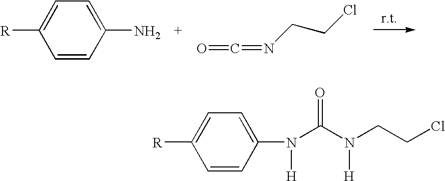

### EXAMPLES 1 to 3

In accordance to the scheme above, the following 3 example molecules were prepared.

| Number | Position, R | R¹ | Yield (%) | MP (° C.) |
|---|---|---|---|---|
| 1 | 4, t-butyl | Chloromethyl | 80 | 133-134 |
| 2 | 4, i-propyl | Chloromethyl | 65 | 140-141 |
| 3 | 4, sec-butyl | Chloromethyl | 95 | 89-90 |

¹H-NMR analysis

| Number | ¹HNMR (200 MHz) (CDCl₃-DMSO-d₆ mixture) |
|---|---|
| 1 | 7.85ppm(1, 1H, ArNH), 7.19ppm(s, 4H, ArH_{2,3,5}, and ₆), |
| | 6.09ppm(broad t, 1H(NHCH₂), 3.51 ppm(m, 4H, |
| | CH₂CH₂Cl), 1.2ppm(s, 9H, t-butyl) |
| 2 | 7.87ppm(1, 1H, ArNH), 7.16(dd, 2H, ArH₂ and ArH₆) |
| | 6.98ppm(dd, 2H, ArH₃ andH₅), 6.06ppm(broad t, 1H(NHCH₂), |
| | 2.71ppm(m, 1H, CH(CH₃)₂, 1.10ppm(dd, 6HCH(CH₃)₂). |
| 3 | 7.80ppm(s, 1H, ArNH), 7.21ppm(dd, 2H, ArH₂ and ArH₆), |
| | 7.00ppm(dd, 2H, ArH₃ andH₄), 6.06ppm(broad t, 1H |
| | (NHCH₂), 3.53ppm,(m, 4H, CH₂CH₂Cl), 2.46ppm(sx, 1H, |
| | CH(CH₃)CH₂CH₃), 1.50ppm(qt, 2H, CH(CH₃)CH₂CH₃), |
| | 1.14ppm(d, 3H, CH(CH₃)CH₂CH₃), 0.74ppm(t, 3H, |
| | CH(CH₃)CH₂CH₃). |

Evaluation of Cytotoxic Activity:

The inventors have surprisingly found clear evidence that the CEUs of the present invention are potent antimicrotubule agents that covalently bind to β-tubulin and consequently prevent microtubule assembly.

To better understand the mechanisms responsible for microtubule disruption by 1-aryl-3(2-chloroethyl)ureas (CEU), their cytotoxicity was examined on Chinese hamster ovary cells resistant to vinblastine and colchicine due to the expression of mutated tubulins (CHO-VV 3-2). These cells showed resistance to CEU, e.g. 4-tBCEU having an IC₅₀ of 21.3±1.1 μM as compared with an IC₅₀ of 11.6±0.7 μM for wild-type cells, suggesting a direct effect of the drugs on tubulins. Western blot analysis confirmed the disruption of microtubules and evidenced the formation of an additional immunoreactive β-tubulin with an apparent lower molecular weight on SDS polyacrylamide gel. Incubation of MDA-MB-231 cells with [urea-¹⁴C]-4-tBCEU revealed the presence of a radioactive protein which coincided with the additional β-tubulin band, indicating that CEU could covalently bind to the β-tubulin. The 4-tBCEU-binding site on β-tubulin was identified by competition of the CEU with colchicine, vinblastine and iodoacetamide, a specific alkylating agent of sulfhydryl groups of cysteine residues. Colchicine, but not vinblastine, prevented formation of the additional β-tubulin band, suggesting that 4-tBCEU alkylates either Cys239 or Cys354 on β-tubulin.

To determine the cysteine residue alkylated by 4-tBCEU, radiolabeled drug was incubated with human neuroblastoma cells (SK—N—SH) which overexpress the βIII-tubulin an isoform where Cys239 is replaced by a serine residue. The results dearly showed that βIII-tubulin is not alkylated by [urea-14C]-4-tBCEU, suggesting that cysteine 239 residue is essential for the reactivity of 4-tBCEU with β-tubulin. Taken together, these findings indicate that the mechanism of cytotoxicity of CEU involves microtubule depolymerization through alkylation of β-tubulin.

### MATERIALS AND METHODS

Cell Culture:

Human breast carcinoma cell line, MDA-MB-231, was obtained from the American Type Culture Collection (ATCC HTB-26; Bethesda, Md.). MDA-MB-231 cells were grown in RPMI 1640 medium supplemented with 10% bovine calf serum (Hyclone, Road Logan, Utah). Wild-type Chinese Hamster Ovary cells (CHO-10001) (7), colchicine- and vinblastine-resistant (CHO-VV 3-2) (8) and taxol-resistant (CHO-TAX 5-6) cells (9) were generously provided by Dr. Fernando Cabral (University of Texas Medical School, Houston, Tex.). These cells were cultured in RPMI 1640 containing 10% fetal bovine serum. SK—N—SH human neuroblastoma cells, (ATCC HTB-11; Bethesda, Md.) were cultured in MEM medium supplemented with 2 mM L-glutamine, 1.5 g/liter sodium bicarbonate, 0.1 mM non-essential amino acids, 1 mM sodium pyruvate and 10% fetal bovine serum. Cells were cultured in a humidified atmosphere at 37° C. in 5% CO₂.

Comparative Drugs:

Colchicine, vinblastine, taxol and iodoacetamide were purchased from Sigma (St Louis, Mo.). CEU derivatives and EBI were prepared as already described (3, 4, 10). Synthesis of [urea-4C]4-tBCEU was carried out as described previously (11). All drugs were dissolved in DMSO and the final concentration of DMSO in the culture medium was maintained at 0.5% (v/v).

Cytotoxiciy Assay:

Cytotoxicity was assessed using the MTT assay (3-(4,5-dimethylthiazol-2-yl)-2,5-diphenyltetrazolium bromide) as described by Carmichael et al. (12). Cytotoxic activity of these compounds was expressed as the concentration of CEU inhibiting MDA-MB-231 cell growth by 50% (IC₅₀).

Kinetics of Alkylation of 4-(4-Nitrobenzyl)Pyridine by CEU Derivatives:

The rate constant of alkylation (K′) of CEU derivatives and chlorambucil was evaluated by a calorimetric assay as described by Bardos et al (13). Briefly, 1 ml of a 10% (v/v) solution of 4-(4-nitrobenzyl)pyridine in ethanol and 1 ml of 50 mM sodium acetate (pH 4.3) were added to an ethanol solution (95%) containing 400 nmol per ml of either chlorambucil or CEU and heated to 80° C. in a shaking water bath for 60, 90, 120 or 150 min. The reaction was stopped by cooling the mixtures on ice for 5 min. Then, 1.5 ml of 0.1 M KOH: ethanol (1:2, v/v) were added to the reaction mixture. Samples were vortexed for 12 sec and set aside for 2.5 min prior to reading the absorbance at 570 nm. The values were compared with those obtained using a blank sample containing all reagents except the alkylating agent.

Cell Cycle Analysis:

Following incubation of MDA-MB-231 cells with 4-tBCEU, 4-iPCEU or 4-sBCEU, the cells were harvested, resuspended in 1 ml PBS and fixed by the addition of 2.4 ml of ice cold anhydrous ethanol. Then, 5×10⁵ cells from each sample were centrifuged for 3 min at 1000×g. Cell pellets were resuspended in PBS containing 50 μg/ml of PI and 40 U/ml of ribonuclease A (Boehringer Mannheim, Laval, Calif.). Mixtures were incubated at room temperature for 30 min and cell cycle distribution was analyzed using an Epics Elite ESP flow cytometer (Coulter Corporation, Miami, Fla., USA).

Separation of Soluble and Polymerized Tubulins:

Separation of soluble and polymerized tubulins from MDA-MB231 cells was carried out as described by Minotti et al. (14) with minor modifications. Briefly, after drug exposure, about 5×10⁶ cells in 100-mm petri dishes were washed with PBS at 37° C. and harvested in 3 ml of PBS containing 0.4 μg/ml of taxol using a rubber policeman.

Cells were centrifuged and lysed using 250 μl of microtubule-stabilizing buffer (20 mM Tris-HCl, pH 6.8, 140 mM NaCl, 1 mM MgCl₂, 2 mM EDTA, 0.5% Nonidet P-40 and 0.4 μg/ml taxol), and then transferred to 1.5 ml microcentrifuge tubes. Samples were centrifuged at 12,000×g for 10 min at 4° C. and the supernatants containing soluble tubulin were placed in separate microcentrifuge tubes containing 250 μl of 2× Laemmli sample buffer (15). Pellets containing the polymerized tubulin were resuspended in 250 μl of water, followed by two freeze/thawing cycles, and the addition of 250 μl of 2× Laemmli sample buffer. Samples were analyzed by SDS-PAGE and immunoassay was performed as described below.

Subcellular Fractionation of MDA-MB-231 Cells.

Cells (˜5×10⁶) were incubated with 30 or 100 μM [¹⁴C]-tBCEU for 12 or 24 h and then washed with PBS and harvested by scraping in lysis buffer (5 mM Hepes pH 7.4, 1 mM MgCl₂, 10 mM KCl, 1 mM CaCl₂, 1 mM PMSF, 1 mM benzamidine and 1 mM aprotinin). Cell lysates were homogenized using a tissue grinder and the final sucrose concentration of each sample was adjusted to 250 mM. Samples were centrifuged at 600×g for 10 min at 4° C. to isolate the post-nuclear supernatant. The pellets, containing nuclei and intact cells, were discarded and the post-nuclear supernatant was recentrifuged at 90,000 rpm using a Rotor TLA-100.1 in a Beckman TL-100 ultracentrifuge for 30 min to separate the cytosolic fraction (C) from the insoluble fraction (M) containing the membrane components and mitochondria. One volume of 2× Laemmli sample buffer was then added to the supernatant (C) and the pellet (M) was resuspended in 200 μl of Laemmli sample buffer. Samples were boiled for 5 min and kept at −20° C. until analysis.

SDS-PAGE Analysis and Immunoblotting of β-Tubulin:

Samples (1×10⁵ cells) were analyzed by 10% SDS-PAGE using the Laemmli system (15). Membranes were then incubated with PBSMT (PBS, pH 7.4, 5% fat-free dry milk and 0.1% Tween-20) for 1 h at room temperature, and then with 1:500 monoclonal anti-β-tubulin (clone TUB 2.1, Sigma) or 1:400 anti (clone no. SDL.3D10, Sigma) for 1 h. This monoclonal antibody is specific to βIII-tubulin, and does not cross-react with other β-tubulin isoforms. Membranes were washed with PBSMT and incubated with 1:2500 peroxidase-conjugated anti-mouse immunoglobulin (Amersham Canada, Oakville, Canada) in PBSMT for 30 min. Detection of the immunoblot was carried out with the ECL Western blotting detection reagent kit (Amersham Canada, Oakville, Canada).

Preparation of Protein Extract and Two-Dimensional SDS Polyacrylamide Gel Electrophoresis:

MDA-MB231 cells (˜5×10⁶) incubated with 30 μM [urea-¹⁴C]-4-tBCEU (11) for 48 h were harvested by scraping and transferred to a 1.5 ml microcentrifuge tube. Cell pellets were lysed by addition of 1 ml lysis buffer containing 4% (w/v) CHAPS, 4.6% Ampholines (comprised of 3.6% Ampholine pH range 5 to 7 and 1% Ampholine pH 3 to 10) (Sigma, St-Louis, Mo.), 50 mM dithiothreitol, 1 mM PMSF and 1 mM benzamidine. Samples were homogenized by 10 passages through a 26 G needle and incubated with 1 mg/ml DNase and 0.25 mg/ml RNase A for 5 min on ice. Then, EDTA/EGTA (1:1) and urea were added to final concentrations of 1 mM and 8.5 M, respectively. Samples were centrifuged at 14 000 rpm for 2 min at 4° C. and 0.03% (w/v) bromophenol blue was added to the supematant Samples were kept at −80° C. until processed. Protein extracts were separated by isoelectric focusing according to the procedure described by O'Farrell (16) with minor modifications. Briefly, samples were applied to a 4.5% polyacrylamide gel containing 8.5 M urea, 2% (w/v) CHAPS and 2% (v/v) ampholines. Samples were prefocused at 200, 300 and 400 Volts for 15, 30 and 30 min respectively (17). Isoelectric focusing was performed at 200, 400, 800 and 600 Volts during 0.5, 15, 1 and 1.5 h, respectively. Gels were equilibrated twice for 15 min in buffer A containing 50 mM Tris-HCl, pH 6.8, 6 M urea, 30% glycerol, 2% (v/v) SDS and 2% (w/v) dithiothreitol and for 10 min in buffer B containing 50 mM Tris-HCl, pH 6.8, 6 M urea, 30% glycerol, 2% (v/v) SDS and 0.5% (w/v) iodoacetamide. In the second dimension, proteins were separated according to their molecular weight by using a 10% polyacrylamide SDS gel. The gels were then transferred onto a nitrocellulose membrane.

Detection of Proteins Alkylated by [urea-¹⁴C]-4-tBCEU:

Nitrocellulose membranes were dried for 3 days at room temperature and fixed under a FBTIV-816 UV transilluminator at 312 nm (Fisher Scientific, Ottawa, Canada) for 3 min. Membranes were incubated for 1 h in Entensify Aqueous Fluor Solution B (DuPont, Boston, Mass.), dried and exposed to X-ray film (Kodak, Biomax MR Film) for a week.

### RESULTS

Structure-Activity Relationships Between the Alkylation Potency and the Cytotoxicity of CEU:

To evaluate the mechanisms responsible for the cytotoxicity of CEU, first was compared the cytotoxicity and the NBP alkylabon constant of various CEU with different IC₅₀ ranging from 2 to >140 μM. Table 1 shows that CEU are weak alkylators of 4-(4-nitrobenzyl)pyridine as compared to chlorambucil (prior art molecule used for comparative purposes).

TABLE 1
Molecular structure, cytotoxic activity and relative alkylation
of 4-(4-nitrobenzyl)pyridine by CEU derivatives.
Concentration inhibiting 50% of cell growth (IC₅₀) as determined
graphically from the survival curves. The constant of alkylation
(K′) was determined by linear regression of the respective
curves generated for each drug. Data represent mean values ± SD
from at least three independent experiments.

| | | | IC₅₀ (μM) | |
|---|---|---|---|---|
| Name | Position, R | R′ | MDA-MB-231 | K′^{b} |
| 4-tBCEU | 4, tBu | —CH₂Cl | 4.6 ± 0.3^{a} | 3.1 ± 0.3^{a} |
| 4-iPCEU | 4, iPr | —CH₂Cl | 2.2 ± 0.3^{a} | 3.0 ± 0.1^{a} |
| 4-sBCEU | 4, sBu | —CH₂Cl | 2.3 ± 0.1^{a} | 3.1 ± 0.3^{a} |
| CBL | Chlorambucil | | 9.3 ± 3.5^{a} | 38.9 ± 5.1^{a} |
| (comparative | | | | |
| example) | | | | |

^{a}Values are mean ± SD.
^{b}K′ = (Aₜ₂ − Aₜ₁/(t₂ − t₁), where A = absorption at 570 nm and (t₂ − t₁) = period of incubation.
^{c}ND, not determined.

As seen from Table 1 above, the K′ of CEU are almost thirteen times lower than those for chlorambucil, a known alkylating agent derived from aromatic nitrogen mustards (18). In addition, the cytotoxicity of different CEU did not correlate with their alkylation potency. Indeed, active CEU such as 4-tBCEU, 4-iPCEU or 4-sBCEU having IC₅₀ of 4, 2 and 2 μM respectively, and inactive CEU such as CEU, 2-ECEU, 4-sBEU and 4-tBCPU, have almost the same K′ values (≈2.5 to 3.5 μM) Furthermore, CEU did not show detectable alkylation of either DNA, glutathione or glutathione reductase. However, substitution of the 2-chloroethyl moiety of active CEU analogs with methyl, ethyl or 3-chloropropyl groups diminished their cytotoxicity. This suggests that albeit very weak, the alkylation potency of CEU is nevertheless involved in the mechanisms of their cytotoxicity.

Differential Cytotoxicity Induced by 4-tBCEU in CHO Cell Lines Expressing Mutated Tubulin:

The cytotoxicity of 4-tBCEU, 4-iPCEU and 4-sBCEU in two CHO cell lines having differential sensitivity to antmicrotubule agents was evaluated. These cell lines are derived from parental CHO-10001 cells and express mutated tubulins. The CHO-VV 3-2 cell line is resistant to vinblastine and colchicine, and hypersensitive to taxol, while the CHO-TAX 5-6 cell line is resistant to taxol and hypersensitive to vinblastine and colchicine (7-9).

Results are reported in Table 2 below.

TABLE 2
Cross-resistance of 4-tBCEU in CHO cells expressing mutated tubulin. Wild-type
(10001), vinblastine and colchicine-resistant (VV 3-2), and taxol-resistant
(TAX 5-6) CHO cells were incubated in the presence of increasing concentrations
of chlorambucil, colchicine, taxol, 4-tBCEU, 4-iPCEU and 4-sBCEU for 120
h. Cell survival was evaluated using the MTT colorimetric assay, 100% survival
representing cell viability in the absence of drug. Data are representative
from three independent experiments.

| | [symbol] | | |
|---|---|---|---|
| Name | CHO 10001 | CHO TAX 5-6 | CHO VV 3-2 |
| Colchicine | 0.0020 ± 0.0003^{a} | 0.00060 ± 0.00001^{a,c} | 0.0036 ± 0.0006^{a,b} |
| Vinblastine | 0.030 ± 0.003^{a} | 0.021 ± 0.003^{a,c} | 0.094 ± 0.003^{a,b} |
| Taxol | 1.7 ± 0.3^{a} | 6.1 ± 0.5^{a,b} | 1.1 ± 0.2^{a,d} |
| 4-tBCEU | 11.6 ± 0.7^{a} | 4.6 ± 0.3^{a,c} | 21.3 ± 1.1^{a,b} |
| 4-iPCEU | 4.4 ± 0.2^{a} | 2.0 ± 0.1^{a,c} | 7.7 ± 0.4^{a,b} |
| 4-sBCEU | 5.0 ± 0.2^{a} | 2.1 ± 0.1^{a,c} | 8.2 ± 0.4^{a,b} |

^{a}Values are mean ± SD of three determinations.
^{b}Significantly more resistant than parental CHO-10001 cells; P ≦ 0.02, Student t-test.
^{c}Significantly more sensitive than parental CHO-10001 cells; P ≦ 0.02, Student t test.
^{d}Not significantly different from parental CHO-10001 cells.

As shown from Table 2, the cytotoxicity of 4-tBCEU, 4-iPCEU and 4-sBCEU was higher in CHO-TAX 5-6 cells (eg. IC₅₀=4.6±0.3 μM for 4-tBCEU) and lower in CHO-VV 3-2 cells (eg. IC₅₀=21.3±1.1 μM for 4-tBCEU). The cytotoxicity of 4-tBCEU in CHO-10001 cells was 11.6±0.7 μM. These results strongly suggest that the cytotoxicity of CEU is due to microtubule depolymerization.

Effects of CEU on MDA-MB-231 Cell Cycle:

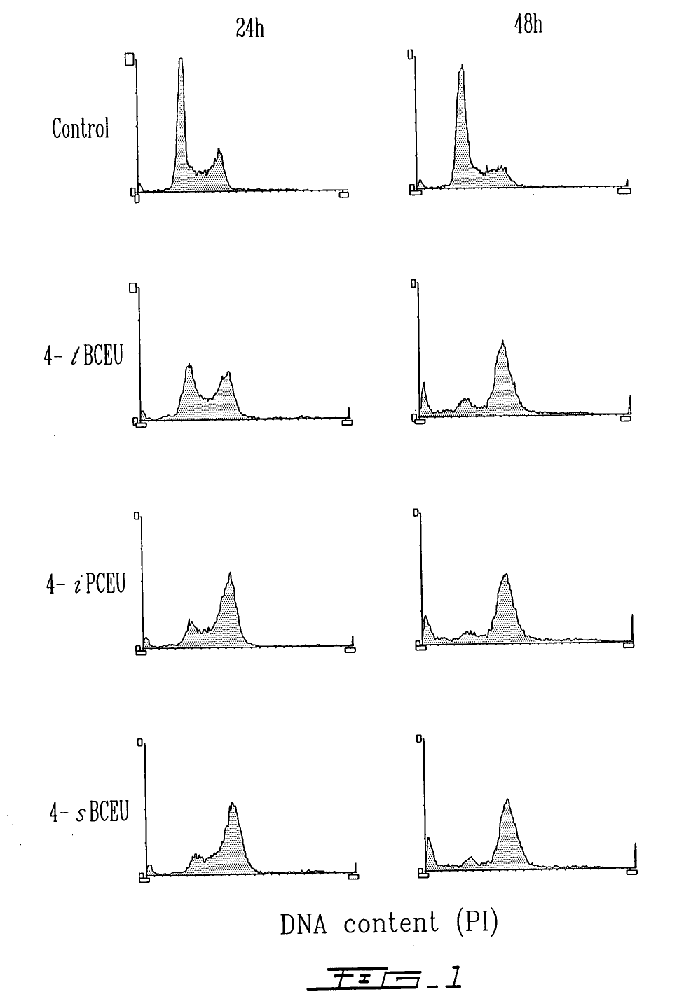

The effect of CEU, namely 4-tBCEU, 4-PCEU and 4-sBCEU, on the cell cycle was analyzed. To this end, exponentially growing MDA-MB-231 cells were treated with 30 μM 4-tBCEU, 4-iPCEU or 4-sBCEU for 24 or 48 h followed by an evaluation of cell cycle distribution by flow cytometry using propidium iodide, a fluorescent DNA dye. This flow cytometric analysis allows to determine the proportion of cells in G₀/G₁, S and G₂+M fractions of the cell cycle but does not allow to distinguish between G₂ and M arrest. As illustrated in FIG. 1, 24 h incubation with CEU derivatives caused a significant accumulation of cells in the G₂+M suggesting that CEU derivatives, like colchicine and vinblastine, induce microtubule disruption and consequently prevent mitosis. Induction of mitosis blockage in prophase by CEU derivatives was confirmed by mitotic index analysis as described by Drouin (22).

Moreover, the cell cycle arrest in G₂+M was induced more rapidly with 4-iPCEU and 4-sBCEU than with 4-tBCEU. As expected, antimicrotubule agents such as colchicine or vinblastine also lead to a G₂+M block in MDA-MB-231 cell cycle.

Effect of CEU on the Depolymerization of Microtubules:

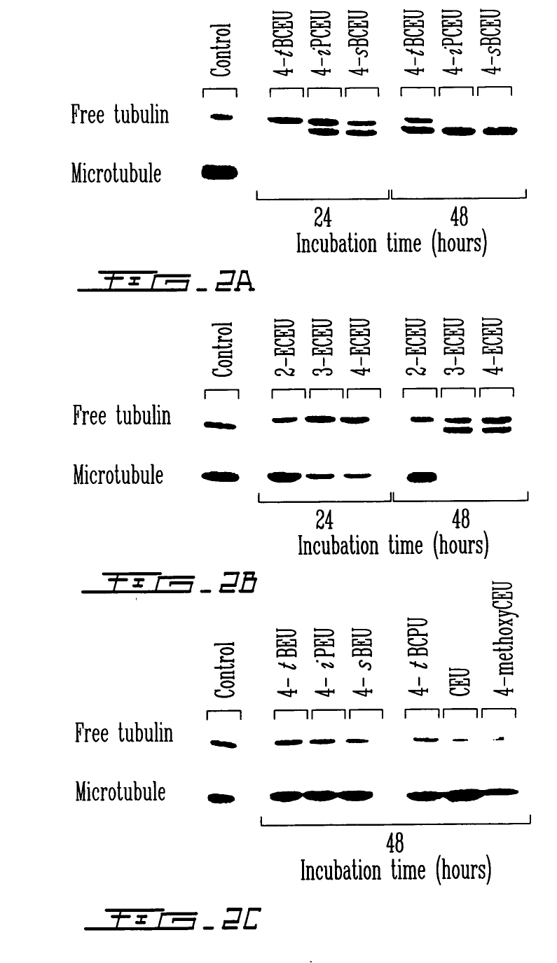

To assess the effects of CEU derivatives on microtubules in MDA-MB-231 cells, the relative levels of polymerized and soluble tubulin in cells using SDS-PAGE analysis and a monoclonal β-tubulin antibody (see Materials and Methods) were determined. FIG. 2A shows that complete microtubule depolymerization was observed after incubation with 30 μM 4-tBCEU, 4-iPCEU or 4-sBCEU for 24 h and 48 h. Furthermore, a second immunoreactive band with an apparent lower molecular weight (Mᵣ) than native β-tubulin was detected. Interestingly, this second immunoreactive band, herein called modified β-tubulin, was only observed in the free tubulin fraction. Formation of the modified β-tubulin was induced in a dose-dependent manner by 4-tBCEU (data not shown). Furthermore, its presence was specific to active CEU, since no such band was observed with colchicine, vinblastine or taxol (data not shown). Finally, the effect of 4-tBCEU seemed to be specific to β-tubulin since no electrophoretic evidence for α-tubulin modification could be found (data not shown). To assess structure-activity relationships between the molecular structure of other CEU derivatives, we compared their relative rates of microtubule depolymerization and formation of the modified β-tubulin. To this end, CEU derivatives were grouped into three sub-classes of activity: i) highly active CEU (IC₅₀ between 2 and 5 μM) (FIG. 2A), ii) weakly active CEU (IC₅₀ between 10 to 50 μM) (FIG. 2B), and iii) essentially inactive CEU (IC₅₀>50 μM) (FIG. 2C). FIG. 2A shows that the effects of the highly active 4-sBCEU and 4-iPCEU on the formation of modified β-tubulin are slighly faster than for 4-tBCEU.

Microtubule depolymerization and the appearance of the modified β-tubulin with CEU of low cytotoxicity such as 3-ECEU and 4ECEU were delayed in time, if compared to the most cytotoxic CEU (FIG. 2A). Moreover, no depolymerization and no effect on β-tubulin were observed with inactive 2-ECEU (FIG. 2B) and with CEU having a modified 2-chloroethyl moiety such as 4-tBEU, 4-iPEU, 4-sBEU (FIG. 2C), suggesting that alkylation is required for β-tubulin inactivation and microtubule disruption. Finally, 4-tBCPU, as well as unsubstituted CEU and CEU bearing polar groups such as 4-methoxy were also inactive (FIG. 2C). When combined these results suggest that alteration of β-tubulin, possibly by alkylation and microtubule disassembly, might at least in part, be responsible for the cytotoxic activity of CEU.

Alkylation of Cellular Proteins by [urea-¹⁴C]-4-tBCEU in MDA-MB-231 Cells:

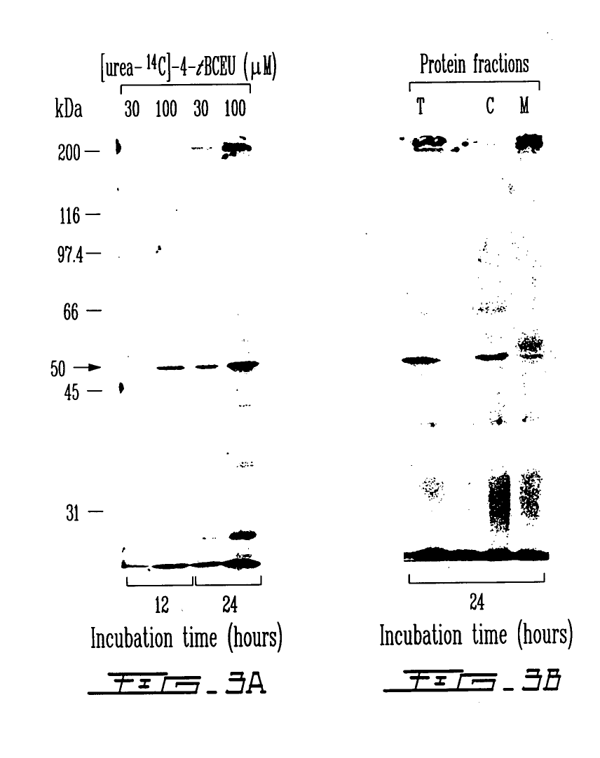

Since the appearance of the modified β-tubulin was the hallmark of active CEU, the inventors determined that CEUs could specifically alkylate β-tubulin. MDA-MB231 cells were incubated with [urea-¹⁴C]-4-tBCEU and cellular proteins were analyzed by SDS-PAGE. In FIG. 3A, a small number of radiolabeled proteins were detected with main radioactive species with Mr at >200, 50, 34 and 29 kDa, respectively. The appearance of these bands was time- and dose-dependent, suggesting that they are specific targets of the radiolabeled 4-tBCEU. Subcellular localization of these proteins indicated that alkylated proteins with Mᵣ at >200 kDa and as well as 34-kDa proteins were found in the insoluble fraction (M) containing the membrane proteins, while the 50-kDa and 29-kDa proteins were found in the cytosolic fraction (C). The protein of 50-kDa is more specifically labeled at low concentrations of [urea-¹⁴C]-4-tBCEU and the molecular weight of this protein band coincided with that of the modified β-tubulin (data not shown), suggesting that 4-tBCEU could alkylate β-tubulin.

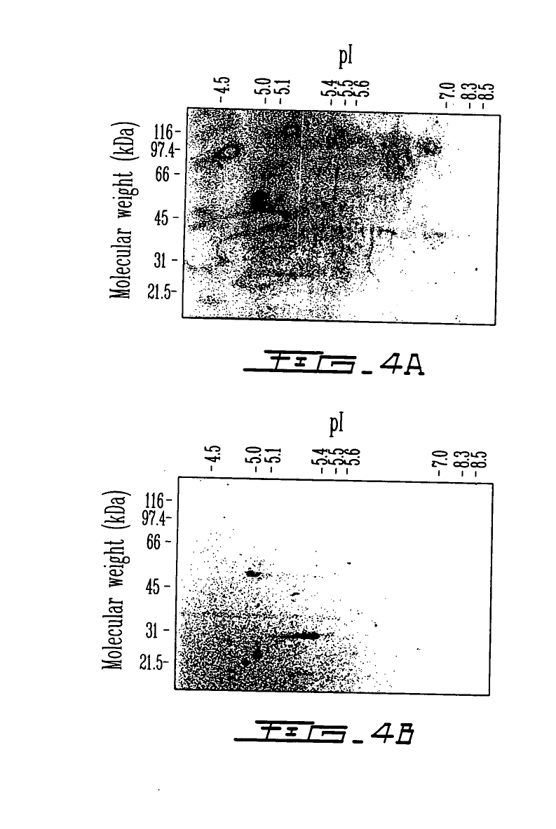

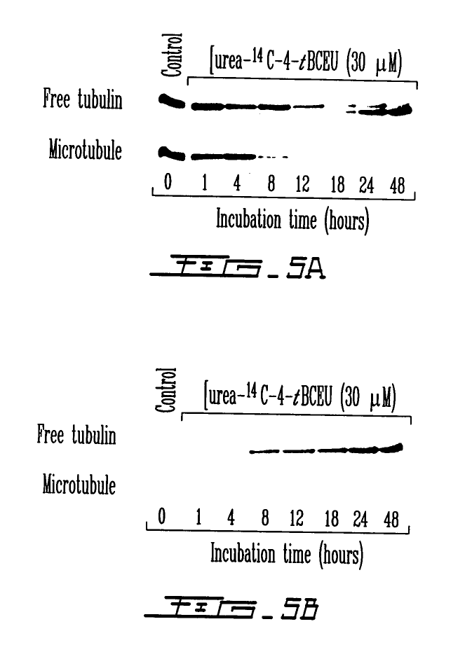

To validate this discovery, the inventors carried out a two-dimensional gel electrophoresis experiment on the proteins extracted from cells treated with 30 μM [urea-¹⁴C]-4-tBCEU for 48 h, (FIGS. 4A and B), while native and modified β-tubulin were revealed by immunoblotting (FIG. 4A). As depicted in FIG. 4B, the ¹⁴C-labeled 50-kDa protein and the modified β-tubulin, co-localized on the gel, indicate that β-tubulin modification likely resulted from direct alkylation by 4-tBCEU. This was confirmed by isolation via two-dimensional gel electrophoresis of the 50-kDa ¹⁴C-labeled protein or modified β-tubulin and mass spectral analysis using matrix-assisted laser desorption/ionization time-of-flight mass spectrometry (23) (data not shown). To determine whether alkylated or modified β-tubulin induce microtubule disassembly, MDA-MB-231 cells were incubated with [urea-¹⁴C]-4-tBCEU for various time intervals, followed by separation of soluble and polymerized tubulins and SDS-PAGE analysis. Native and modified β-tubulin were detected by a monoclonal β-tubulin antibody (FIG. 5A) and whereas ¹⁴C-labeled modified β-tubulin was detected by autoradiography (FIG. 5B). FIG. 5A shows that complete microtubule disruption occurs after 8 to 12 h of incubation whereas modified β-tubulin appears only at 12 h. Interestingly, FIG. 5B illustrates that ¹⁴C-labeled modified β-tubulin appears after only 1 to 4 h of incubation with the drug. These results confirm that the alkylation of β-tubulin occurs prior to microtubule disruption, suggesting that β-tubulin alkylation by [urea-¹⁴C]-4-tBCEU induces microtubule disassembly. Interestingly, alkylated β-tubulin was found solely in the soluble protein fraction, suggesting that the alkylation of β-tubulin is possible only when the protein is in its unpolymerized state. Thus, the site of β-tubulin alkylation by CEU might be involved in the microtubule assembly process, through as yet unknown mechanisms.

Identification of the Alkylation Site of CEU on β-Tubulin:

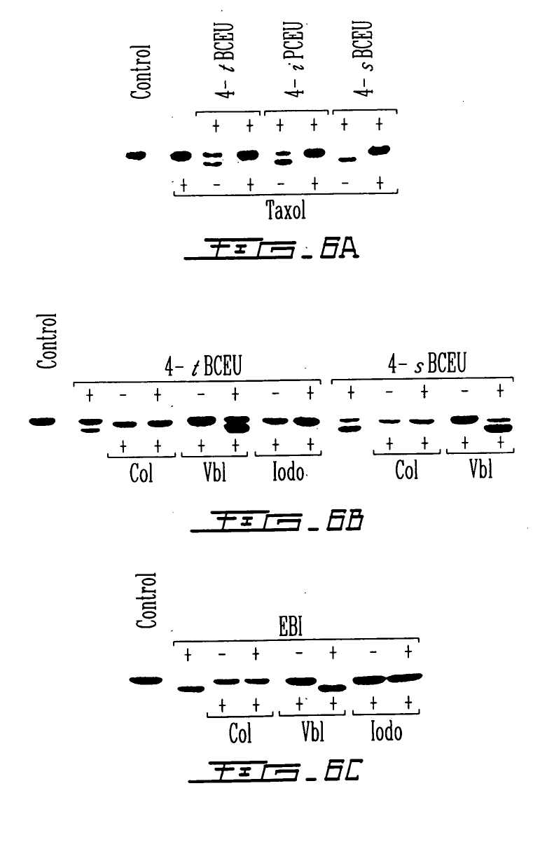

To identify the site of β-tubulin alkylation by CEU, competition experiments between 4-tBCEU, 4-iPCEU, 4-sBCEU and various antimicrotubule agents such as taxol, colchicine and vinblastine were carried out. The latter agents were used because they have well defined binding sites on β-tubulin (24-28) and because they were expected to inhibit alkylation by CEU if they shared a common binding site. FIG. 6A shows that treatment of MDA-MB-231 cells with 4-tBCEU, 4-iPCEU or 4-sBCEU in the presence of taxol, a microtubule stabilizing agent (29, 30), abrogated the formation of modified β-tubulin. Furthermore, immunoblotting experiments (FIG. 6B) showed that colchicine, but not vinblastine, prevented the formation of modified β-tubulin, induced by 4-tBCEU or 4-sBCEU. These data suggest that the binding site of CEU on β-tubulin is in the vicinity of the colchicine binding site. This was confirmed by treatment of cells with [urea-¹⁴C]-4-tBCEU in presence of colchicine or vinblastine, which showed the inhibition of the formation of the 50-kDa radiolabeled band by the former, but not the latter drug (data not shown). Two potential sites of alkylation have been identified in the vicinity of the colchicine binding site on β-tubulin (25). These putative alkylation sites are two nucleophilic mercapto groups of cysteine residues at positions 239 and 354, respectively (25). To assess the possible alkylation of these mercapto residues by CEU, MDA-MB-231 cells were treated with 4-tBCEU in the presence of iodoacetamide, a specific thiol alkylating agent (31, 32). FIG. 6B shows that iodoacetamide completely prevented β-tubulin alkylation by 4-tBCEU, indicating that 4-tBCEU alkylates cysteine residues. Similar experiments were carried out using EBI, a bifunctional alkylating agent that specifically induces cross-linking between Cys239 and Cys354 of β-tubulin (32).

This modification altered the electrophoretic behaviour of β-tubulin (32) similar to the modification observed with cytotoxic CEU. Interestingly, the β-tubulin alteration induced by EBI is abrogated by pre-treatment of cells with colchicine or iodoacetamide but not with vinblastine (FIG. 6C). These results strongly suggest that EBI and CEU share the same alkylation site on β-tubulin, which is either Cys239 or Cys354 or both.

Localization of the Cysteine Residue(s) Alkylated by CEU:

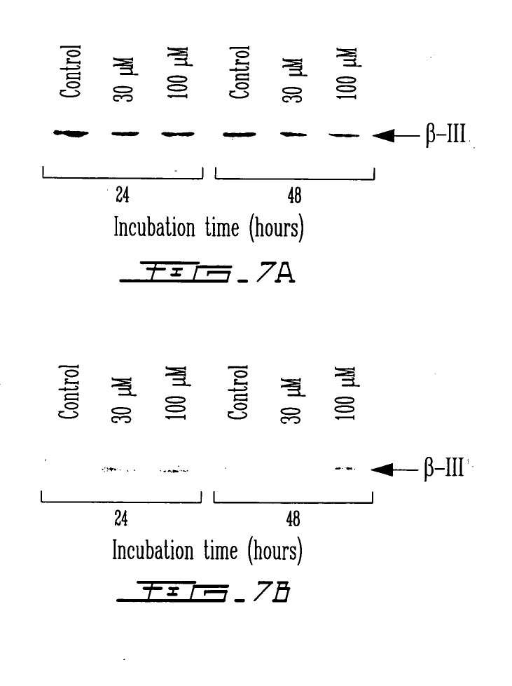

The results presented above suggest that alkylation of cysteine residues of β-tubulin could occur either on Cys239 or Cys354 or on both residues. Alkylation of both residues is most unlikely since CEU are monoalkylating agents. To discriminate between alkylation of Cys239 or Cys354, we compared the relative alkylation induced by [urea-¹⁴C]-4-tBCEU on βIII-tubulin isoform (FIG. 7A) using an antibody which specifically recognizes βIII-tubulin without cross-reactivity with other tubulin isotypes. βIII-tubulin, is characterized by the substitution of the Cys239 residue by a serine residue. In this case, serine residues are not nucleophilic enough to be alkylated by a soft alkylating agent such as [urea-¹⁴C]-4-tBCEU. SK—N—SH cells, which express significant amounts of neuronal-specific βIII-tubulin (33) as well as several other isoforms such as βI-, βII- and βIV-tubulin, were treated with [urea-¹⁴C]-4-tBCEU. In contrast with other β-tubulin isoforms containing Cys239 (cf. FIG. 5), FIG. 7A shows that [urea-¹⁴C]-4-tBCEU does not alter migration of the β-tubulin on SDS-PAGE and does not decrease cytosolic levels of βIII-tubulin. Moreover, the autoradiogram (FIG. 7B) shows that βIII-tubulin is not alkylated by [urea-¹⁴C]-4-tBCEU since βIII-tubulin and the ¹⁴C-labeled band are not co-localized on the gel. The ¹⁴C-labeled band observed in FIG. 7B corresponds to other β-tubulin isoforms such as βI-, βII- and βIV-tubulin. Taken together, these results suggest that the residue alkylated by CEU is most likely Cys239.

Findings

The present inventors have discovered the β-tubulin inhibiting properties of certain CEU's. Furthermore, it was discovered that these CEUs do not significantly alkylate nucleophiles such as DNA, glutathione and glutathione reductase, which are targeted by most commerically used alkylating agents such as nitrogen and phosphoramide mustards, nitrosoureas, methanesulfonate esters and aziridines (34, 35). The present invention shows that the cytotoxicity of these CEUs and their ability to selectively alkylate β-tubulin requires both weak alkylating properties and a hydrophobic character.

According to our structure-activity relationship studies, the aryl-3-(2-chloroethyl)urea moiety was found to be the pharmacophore responsible for the soft alkylating properties of CEU. The second portion of the molecule referred to as its “prosthetic moiety”, is responsible for the hydrophobic properties of CEU and seems of utmost importance for the pharmacological activity of CEU on β-tubulin. Indeed, the pharmacophore per se is non-cytotoxic, whereas substitution of the aromatic ring at the 4-position by lower alkyl groups leads to cytotoxic CEU derivatives able to specifically alkylate β-tubulin. The kinetics of alkylation of β-tubulin suggest that nucleophilic addition of CEU requires a relatively long period of incubation. This type of kinetic behavior is probably related to several factors such as slow diffusion of the drugs into the cytosol and their weak alkylating properties, leading to slow nucleophilic addition. Nevertheless, the covalent binding of CEU to proteins seems specific and irreversible.

Furthermore, in the case of the in vivo alkylation of β-tubulin, the protein must be under its depolymerized form to react with CEU. The β-tubulin monomer, once alkylated, becomes incompetent for microtubule formation.

In a preferred embodiment, it was determined that the most likely reactive site of 4-tBCEU was either Cys239 or Cys354, in the vicinity of the colchicine-binding site, since colchicine inhibits β-tubulin alkylabon by CEU. Moreover, it was demonstrated that β-tubulin with a Cys_{→}Ser substitution at position 239 is not alkylated by the drug, suggesting that Cys239 might be the residue alkylated by a CEU such as 4-tBCEU. Previous evidence had established that Cys239, but not Cys354, is specifically alkylated by synthetic compounds such as 2,4-dichlorobenzyl thiocyanate (36) and 2-fluoro-1-methoxy-4-pentafluorophenyl-sulfonamidobenzene (37) inducing microtubule disassembly. These results suggest that Cys239 is more sensitive and more accessible to alkylation than Cys354. Thus, the integrity of Cys239 is most likely essential in the microtubule assembly process. However, the inventors cannot discard the possibility that 4-tBCEU alkylates β-tubulin at other residues on the protein, and that Cys239 is essential to maintain the proper conformation of β-tubulin reactive with CEU. Alkylation of β-tubulin by CEU induces the formation of a modified β-tubulin, which migrates ahead of native β-tubulin on SDS-PAGE. The electrophoretic behaviour of the modified β-tubulin obtained by alkylation of β-tubulin by CEU, is similar to the modified β-tubulin observed after the formation of a cross-link between Cys239 and Cys354 by EBI (32). It is important to mention that CEU are monoalkylating agents and are therefore unlikely to induce such cross-links in β-tubulin.

The probability that CEU could carbamoylate proteins through reaction of the carbonyl group of the urea moiety with lysine or cysteine residues in the vicinity of Cys239 or Cys354 is most unlikely. The chemical stability of aromatic ureas is very high and does not allow nucleophilic reactions, even with highly nucleophilic entities such as glutathione and glutathione reductase (data not shown). Moreover, there are no other nucleophilic entities present, either in the hydrophobic pocket or in the vicinity of the hydrophobic pocket that are available for such a reaction (25).

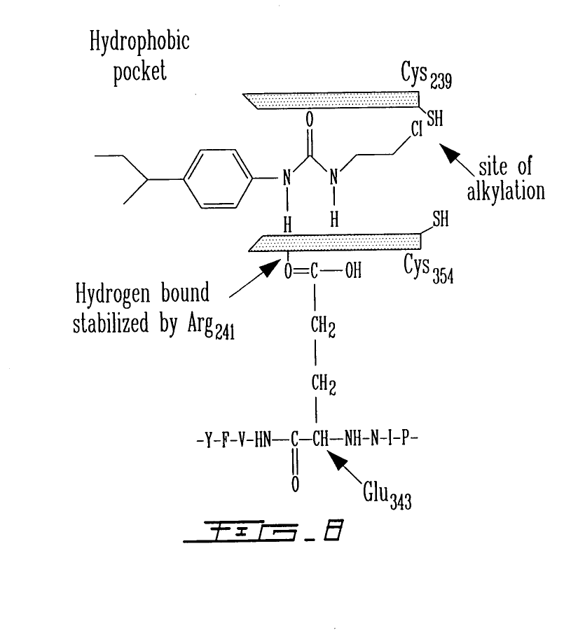

A plausible mechanistic explanation to the formation of modified β-tubulin by CEU is illustrated in FIG. 8. This putative mechanism of alkylation of β-tubulin by CEU is based on the analysis of the three-dimensional structure of β-tubulin, recently published by Nogales (25) and on the results of the present study. Briefly, the nucleophilic Cys239 is present in the drug-binding domain of colchicine, which is delimited by residues 206-381 that containing four mixed β-sheets and five surrounding α-helices. Two of the β-sheets, β8 and β9, are in dose proximity, leading to the formation of a hydrophobic pocket (˜8 Å across, between Cys239 and Cys354) (25). The existence of a hydrophobic pocket in tubulin was previously suggested as being in the vicinity of the colchicine binding site (38). This hydrophobic cavity could accommodate the aromatic ring, substituted by lower alkyl groups at the 4-position. It is speculated that the hydrophobic moiety of CEU readily forms hydrophobic bonds with several amino acid residues such as valine (316, 342, 349, 353), alanine (314, 315, 352) and leucine (240), present in the cavity. In addition, a strong hydrogen bond might be formed between the aromatic amino group of CEU and the glutamic acid residue at position 343. This hydrogen bond could be stabilized by electron resonance using the arginine residue at position 241.

After docking the CEU in the hydrophobic pocket, a hydrogen bond with the glutamic acid residue at position 343 would be formed; followed by an alkylation between the Cys239 residue and the 2-chloroethylamino moiety of CEU.

In summary, CEU are weak monoalkylating agents that are unreactive toward most cellular nucleophiles such as DNA, glutathion and glutathion reductase. On the other hand, CEUs were shown to alkylate specific cancer cell proteins bearing strong nucleophilic centers that present a spatial environment favoring close and prolonged contacts between the drug and the nucleophilic moiety.

These elements describe the concept of “soft alkylation”, which introduces new perspectives about the rational design of drugs that might be able to inactivate specific cellular proteins with resulting cytotoxic effects directed at tumor cells.

### REFERENCES

1. C.-Gaudreault, R., Lacroix, J., Pagé, M., and Joly, L. P. 1-aryl3-(2-chloroethyl)ureas: synthesis and in vitro assay as potential anticancer agents, J Pharm Sci. 77: 185-187, 1988.
2. Lacroix, J., C.-Gaudreault, R., Pagé, M., and Joly, L. P. In vitro and in vivo activity of 1-aryl-3-(2-chloroethyl)urea derivatives as new antineoplastic agents, Anticancer Res. 8: 595-8, 1988.
3. Béchard, P., Lacroix, J., Poyet, P., and C.-Gaudreault, R. Synthesis and cytotoxic activity of new alky-[3-(2-chloroethyl)ureido]benzene derivatives, Eur. J. Med. Chem. 29: 963-966, 1994.
4. C.-Gaudreault, R., Alaoui-Jamali, M. A., Batist, G., Béchard, P., Lacroix, J., and Poyet, P. Lack of cross-resistance to a new cytotoxic arylchloroethyl urea in various drug-resistant tumor cells, Cancer Chemother. Pharmacol. 33: 489-92, 1994.
5. Geran, G. I., Greenberg, N. H., Macdonald, N. M., Schumacher, A. M., and Abbott, B. J. Protocols for screening chemical agents and natural products against animal tumors and other biological (third edition), Cancer Chemother. Rep. Part 3, vol 3: 1-103, 1972.
6. Poyet, P., Ritchot, N., Béchard, P., and C.-Gaudreault, R. Effect of an aryl chloroethyl urea on tubulin and vimentin syntheses in a human breast cancer cell line, Anticancer Res. 13: 1447-1452, 1993.
7. Cabral, F., Sobel, M. E., and Gottesman, M. M. CHO mutants resistant to colchicine, colcemid or griseofulvin have an altered b-tubulin., Cell. 20: 29-36, 1980.
8. Schibler, M. J., Barlow, S. B., and Cabral, F. Elimination of permeability mutants from selections for drug resistance in mammalian cells, FASEB J. 3: 163-168, 1989.
9. Schibler, M. J. and Cabral, F. Taxol-dependent mutants of Chinese hamster ovary cells with alterations in alpha- and beta-tubulin, J Cell Biol. 102: 1522-1531, 1986.
10. Luduena, R. F., Roach, M. C., Trck, P. P., Little, M., Palanivelu, P., Binkley, P., and Prasad, V. b2-tubulin, a form of chordate brain tubulin with lesser reactivity toward an assembly-inhibition sulfhydryl-directed crosslinking reagent, Biochemistry. 21: 4787-4794, 1982.
11. Azim, M., Madelmont, J. C., Cussac, C., Rapp, M., Maurizis, J. C., C.-Gaudreault, R., Godeneche, D., and Veyre, A. Marquage par 14C et 13C du tert-butylanilino4-chloroéthylurée, J Labelled Compd. Rad. 33: 1079-1082, 1993.
12. Carmichael, J., De Graff, W. G., Gazdar, A. F., Minna, l. D., and Mitchell, J. B. Evaluation of a tetrazolium-based semi-automated calorimetric assay assessment of chemosensitivity testing, Cancer Res. 47: 943-946, 1987.
13. Bardos, T., Datta-Gupta, P., Hebborn, P., and Triggle, D. J. Study of comparison chemical and biological activities of alkylating agents. J Med Chem. 8: 167, 1965.
14. Minotti, A. M., Barlow, S. B., and Cabral, F. Resistance to antimitotic drugs in chinese hamster ovary cells correlates with changes in the level of polymerized tubulin, J Biol Chem. 266: 3987-3994, 1991.
15. Laemmli, U. K. Cleavage of structural proteins during assembly of the head of bacteriophage T4, Nature. 227: 680-685, 1970.
16. O'Farrell, P. H. High Resolution Two-Dimensional Electrophoresis of Proteins, J. Biol. Chem. 250: 4007-4021, 1975.
17. Lognonné, J.-L. 2D-PAGE analysis: a pratical guide to principle critical parameters. Cell Mol Biol. 40: 41-55, 1994.
18. Povirk, L. F. and Shuker, D. E. DNA damage and mutagenesis induced by nitrogen mustards, Mutation Research. 318: 205-226, 1994.
19. Ben-Ze'ev, A., Farmer, S. R., and Penman, S. Mechanisms of regulating tubulin synthesis in cultured mammalian cells, Cell. 17: 319-325, 1979.
20. Wendell, K. L., Wilson, L., and Jordan, M. A. Mitotic block in HeLa cells by vinblastine: ultrastructural changes in kinetocore-microtubule attachment and in centrosomes, J Cell Sci. 104: 261-274, 1993.
21. Wilson, L. and Jordan, M. A. Microtubule dynamics: taking aim at a moving target, Chem Biol. 2: 569-573, 1995.
22. Drouin, R., Lemieux, N., and Richer, C.-L. High-resolution R-banding at the 1250-band level. I. Technical considerations on cell synchronization and R-banding (RHG and RBG), Cytobios. 56: 107-125, 1988.
23. Sechi, S. and Chait, B. T. Modification of cysteine residues by alkylation. A tool in peptide mapping and protein identification. Anal. Chem. 70: 5150-5158, 1998.
24. Parekh, H. and Simpkins, H. The transport and binding of taxol, Gen Pharmacol. 29: 167-172, 1997.
25. Nogales, E., Wolf, S. G., and Downing, K. H. Structure of the ab tubulin dimer by electron crystallography, Nature. 391: 199-203, 1998.
26. Burns, R. G. Analysis of the colchicine-binding site of beta-tubulin, FEBS Lett. 297: 205-208, 1992.
27. Uppuluri, S., Knipling, L., Sackett, D. L., and Wolff, J. Localization of the colchicine-binding site of tubulin, Proc Nati Acad Sci USA. 90: 11598-11598, 1994.
28. Lobert S., Frankfurter, A., and Correia, J. J. Binding of vinblastine to phosphocellulose-purified and alphabeta-class III tubulin: The role of nucleotides and beta-tubulin isotypes, Biochemistry. 34: 8050-8060, 1995.
29. Schiff, P. B., Fant, J., and Horwitz, S. B. Promotion of microtubule assembly in vitro by taxol, Nature. 277: 665-667, 1979.
30. Schiff, P. B. and Horwitz, S. B. Taxol stabilizes microtubules in mouse fibroblast cells, Proc Nag Acad Sci USA. 77: 1561-1565, 1980.
31. Luduena, R. F. and Roach, M. C. Interaction of tubulin with drugs and alkylating agents. 1. Alkylabon of tubulin by iodo[¹⁴C]acetamide and N,N′-ethylenebis-(iodoacetamide), Biochemistry. 20: 4437-4444, 1981.
32. Luduena, R. F. and Roach, M. C. Tubulin sulfhydryl groups as probes and targets for antimitotic and antimicrotubule agents, Pharmac Ther. 49: 133-152, 1991.
33. Lee, M. K, Rebhun, L. I., and Frankfurter, A. Posttranslational modification of class III b-tubulin, Proc Natl Acad Sci USA. 87: 7195-7199, 1990.
34. Sanderson, B. J. and Shield, A. J. Mutagenic damage to mammalian cells by therapeutic alkylating agents, Mutaffon Research. 355: 41-57, 1996.
35. Dong, Q., Barsky, D., Colvin, M. E., Melius, C. F., Ludeman, S. M., Moravek, J. F., Colvin, O. M., Bigner, D. D., Modrich, P., and Friedman, H. S. A structural basis for a phosphoramide mustard-induced DNA interstrand cross-link at 5′-d(GAC), Proc Natl Acad Sci USA. 92: 12170-12174, 1995.
36. Bai, R.-L., Lin, C. M., Nguyen, N. Y., Liu, T.-Y., and Hamel, E. Identification of the cysteine residue of b-tubulin alkylated by the antimitotic agent 2,4-dichlorobenzyl thiocyanate, facilited by separation of the protein subunits of tubulin by hydrophobic column chromatography, Biochemistry. 28: 5606-5612, 1989.
37. Shan, B., Medina, J. C., Santha, E., Frankmoelle, W. P., Chou, T.-C., Learned, R. M., Narbut, M. R., Stott, D., Wu, P., Jaen, J. C., Rosen, T., Timmermans, P. B. M. W. M., and Beckmann, H. Selective, covalent modification of b-tubulin residue Cys-239 by T138067, an antitumor agent with in vivo efficacy against multidrug-resistant tumors, Proc. Natl. Acad. Sci. USA. 96: 5686-5691, 1999.
38. Deinum, J. and Lincoln, P. Binding to tubulin of an allocolchicine spin probe: interaction with the essential SH groups and other active sites, Biochimica et Biophysica Acta. 870: 226-233, 1986.

1. (canceled)

2. (canceled)

3. (canceled)

4. (canceled)

5. (canceled)

6. (canceled)

7. (canceled)

8. A method for selectively alkylating β-tubulin, said method comprising:
i) providing a molecule of formula I, or a pharmaceutically acceptable salt thereof: 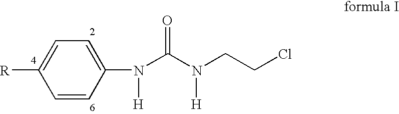
wherein R is selected from the group consisting of t-butyl, i-propyl and s-butyl; and
ii) contacting cells with the compound of formula I,
wherein said contacting of said cells alkylates β-tubulin.

9. The method of claim 8, wherein said cells are from a human breast carcinoma.
